SEQUENCE LISTING (27 sequences)

SEQ ID NO: 1 - protein, 101 aa, Homo sapiens
MAMMEVQGGPSLGQTCVLIVIFTVLLQSLCVAVTYVYFTNELKQMQDKYSKSGIACFLKEDDSYWDPNDEESMNSPCWQVKWQLRQLVRKTPRMKRLWAAK

SEQ ID NO: 2 - protein, 11 aa, Homo sapiens
TPRMKRLWAAK

SEQ ID NO: 3 - DNA, 306 bp, Homo sapiens
  source 1..306: mol_type = other DNA
atggctatgatggaggtccaggggggacccagcctgggacagacctgcgtgctgatcgtgatcttcacagtgctcctgcagtctctctgtgtggctgtaacttacgtgtactttaccaacgagctgaagcagatgcaggacaagtactccaaaagtggcattgcttgtttcttaaaagaagatgacagttattgggaccccaatgacgaagagagtatgaacagcccctgctggcaagtcaagtggcaactccgtcagctcgttagaaagactccaagaatgaaaaggctctgggccgcaaaataa

SEQ ID NO: 4 - protein, 63 aa, Homo sapiens
TNELKQMQDKYSKSGIACFLKEDDSYWDPNDEESMNSPCWQVKWQLRQLVRKTPRMKRLWAAK

SEQ ID NO: 5 - protein, 101 aa, synthetic construct
MAMMEVQGGPSLGQTCVLIVIFTVLLQSLCVAVTYVYFTNELRQMQDKYSKSGIACFLKEDDSYWDPNDEESMNSPCWQVKWQLRQLVRKTPRMKRLWAAK

SEQ ID NO: 6 - protein, 101 aa, synthetic construct
MAMMEVQGGPSLGQTCVLIVIFTVLLQSLCVAVTYVYFTNELKQMQDKYSKSGIACFLKEDDSYWDPNDEESMNSPCWQVKWQLKQLVRKTPRMKRLWAAK

SEQ ID NO: 7 - protein, 101 aa, synthetic construct
MAMMEVQGGPSLGQTCVLIVIFTVLLQSLCVAVTYVYFTNELKQMQDKYSKSGIACFLKEDDSYWDPNDEESMNSPCWQVHWQLRQLVRKTPRMKRLWAAK

SEQ ID NO: 8 - protein, 101 aa, synthetic construct
MAMMEVQGGPSLGQTCVLIVIFTVLLQSLCVAVTYVYFTNELKQMQDKYSKSGIACFLKEDDSYWDPNDEESMNSPCWQVHWQLKQLVRKTPRMKRLWAAK

SEQ ID NO: 9 - protein, 101 aa, synthetic construct
MAMMEVQGGPSLGQTCVLIVIFTVLLQSLCVAVTYVYFTNELRQMQDKYSKSGIACFLKEDDSYWDPNDEESMNSPCWQVHWQLKQLVRKTPRMKRLWAAK

SEQ ID NO: 10 - protein, 101 aa, synthetic construct
MAMMEVQGGPSLGQTCVLIVIFTVLLQSLCVAVTYVYFTNELRQMQDKYSKSGIACFLKEDDSYWDPNDEESMNSPCWQVKWQLKQLVRKTPRMKRLWAAK

SEQ ID NO: 11 - protein, 101 aa, synthetic construct
MAMMEVQGGPSLGQTCVLIVIFTVLLQSLCVAVTYVYFTNELRQMQDKYSKSGIACFLKEDDSYWDPNDEESMNSPCWQVHWQLRQLVRKTPRMKRLWAAK

SEQ ID NO: 12 - protein, 63 aa, synthetic construct
TNELRQMQDKYSKSGIACFLKEDDSYWDPNDEESMNSPCWQVKWQLRQLVRKTPRMKRLWAAK

SEQ ID NO: 13 - protein, 63 aa, synthetic construct
TNELKQMQDKYSKSGIACFLKEDDSYWDPNDEESMNSPCWQVKWQLKQLVRKTPRMKRLWAAK

SEQ ID NO: 14 - protein, 63 aa, synthetic construct
TNELKQMQDKYSKSGIACFLKEDDSYWDPNDEESMNSPCWQVHWQLRQLVRKTPRMKRLWAAK

SEQ ID NO: 15 - protein, 63 aa, synthetic construct
TNELKQMQDKYSKSGIACFLKEDDSYWDPNDEESMNSPCWQVHWQLKQLVRKTPRMKRLWAAK

SEQ ID NO: 16 - protein, 63 aa, synthetic construct
TNELRQMQDKYSKSGIACFLKEDDSYWDPNDEESMNSPCWQVHWQLKQLVRKTPRMKRLWAAK

SEQ ID NO: 17 - protein, 63 aa, synthetic construct
TNELRQMQDKYSKSGIACFLKEDDSYWDPNDEESMNSPCWQVKWQLKQLVRKTPRMKRLWAAK

SEQ ID NO: 18 - protein, 63 aa, synthetic construct
TNELRQMQDKYSKSGIACFLKEDDSYWDPNDEESMNSPCWQVHWQLRQLVRKTPRMKRLWAAK

SEQ ID NO: 19 - protein, 298 aa, synthetic construct
MTNELKQMQDKYSKSGIACFLKEDDSYWDPNDEESMNSPCWQVKWQLRQLVRKTPRMKRLWAAKYSSTMVRSYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 20 - protein, 298 aa, synthetic construct
MYSSTMVRSYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGKTNELKQMQDKYSKSGIACFLKEDDSYWDPNDEESMNSPCWQVKWQLRQLVRKTPRMKRLWAAK

SEQ ID NO: 21 - protein, 336 aa, synthetic construct
MAMMEVQGGPSLGQTCVLIVIFTVLLQSLCVAVTYVYFTNELKQMQDKYSKSGIACFLKEDDSYWDPNDEESMNSPCWQVKWQLRQLVRKTPRMKRLWAAKMYSSTMVRSYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 22 - protein, 336 aa, synthetic construct
MYSSTMVRSYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGKMAMMEVQGGPSLGQTCVLIVIFTVLLQSLCVAVTYVYFTNELKQMQDKYSKSGIACFLKEDDSYWDPNDEESMNSPCWQVKWQLRQLVRKTPRMKRLWAAK

SEQ ID NO: 23 - protein, 298 aa, synthetic construct
TNELKQMQDKYSKSGIACFLKEDDSYWDPNDEESMNSPCWQVKWQLRQLVRKTPRMKRLWAAKMYSSTMVRSYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 24 - protein, 288 aa, synthetic construct
MAMMEVQGGPSLGQTCVLIVIFTVLLQSLCVAVTYVYFTNELKQMQDKYSKSGIACFLKEDDSYWDPNDEESMNSPCWQVKWQLRQLVRKTPRMKRLWAAKMTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 25 - protein, 287 aa, synthetic construct
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRNMAMMEVQGGPSLGQTCVLIVIFTVLLQSLCVAVTYVYFTNELKQMDKYSKSGIACFLKEDDSYWDPNDEESMNSPCWQVKWQLRQLVRKTPRMKRLWAAK

SEQ ID NO: 26 - protein, 250 aa, synthetic construct
TNELKQMQDKYSKSGIACFLKEDDSYWDPNDEESMNSPCWQVKWQLRQLVRKTPRMKRLWAAKMTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 27 - protein, 250 aa, synthetic construct
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRNTNELKQMQDKYSKSGIACFLKEDDSYWDPNDEESMNSPCWQVKWQLRQLVRKTPRMKRLWAAK

(19) United States
(12) Patent Application Publication
(10) Pub. No.: US 2025/0295730 A1
(43) Pub. Date: Sep. 25, 2025

(54) METHODS AND MATERIALS FOR MODULATING T CELL ACTIVATION USING TRAILshort POLYPEPTIDES

(71) Applicant: Mayo Foundation for Medical Education and Research, Rochester, MN (US)

(72) Inventor: Andrew D. Badley, Rochester, MN (US)

(21) Appl. No.: 18/868,869

(22) PCT Filed: Jun. 1, 2023
(86) PCT No.: PCT/US2023/024097

§ 371 Date: Nov. 25, 2024

Related U.S. Application Data
(60) Provisional application No. 63/348,620, filed on Jun. 3, 2022.

(51) Int. Cl. A61K 38/16; A61K 45/06; A61P 37/06; C07K 16/28

(52) U.S. Cl. CPC A61K 38/16; A61K 45/06; A61P 37/06; C07K 16/2875

(57) ABSTRACT

This document relates to methods and materials for modulating (e.g., reducing) T cell activation (e.g., T cell proliferation, T cell effector functions, and/or cytokine secretion) using a TRAILshort polypeptide, a variant TRAILshort polypeptide, or a TRAILshort fusion polypeptide. For example, a TRAILshort polypeptide, a variant TRAILshort polypeptide, or a TRAILshort fusion polypeptide can be administered to a mammal in need thereof, e.g., a human with an autoimmune disorder, graft versus host disease, lichen planus or other disorder characterized by excessive T cell activation, to reduce excessive T cell activation (e.g., excessive T cell proliferation, excessive T cell effector functions, and/or excessive cytokine secretion).

### CROSS-REFERENCE TO RELATED APPLICATIONS

This application claims priority from U.S. Provisional Application Ser. No. 63/348,620, filed Jun. 3, 2022. The disclosure of the prior application is considered part of (and is incorporated by reference in) the disclosure of this application.

### STATEMENT OF FEDERALLY SPONSORED RESEARCH

This invention was made with government support under AI110173 and AI120698 awarded by the National Institutes of Health. The government has certain rights in the invention.

### SEQUENCE LISTING

This application contains a Sequence Listing that has been submitted electronically as an XML file named “07039-2113WO1.XML.” The XML file, created on May 30, 2023 is 28,468 bytes in size. The material in the XML file is hereby incorporated by reference in its entirety.

### BACKGROUND

### 1. Technical Field

This document relates to methods and materials for modulating (e.g., reducing) T cell activation (e.g., T cell proliferation, T cell effector functions, and/or cytokine secretion) using a TRAILshort polypeptide, a variant TRAILshort polypeptide, or a TRAILshort peptidomimetic. For example, a TRAILshort polypeptide, a variant TRAILshort polypeptide, or a TRAILshort peptidomimetic can be administered to a mammal in need thereof, e.g., a human with an autoimmune disorder, graft versus host disease, lichen planus or other disorder characterized by excessive T cell activation, to reduce excessive T cell activation (e.g., excessive T cell proliferation, excessive T cell effector functions, and/or excessive cytokine secretion). This document also provides compositions containing particles (e.g., microparticles and/or nanoparticles) designed to include a TRAILshort polypeptide, a variant TRAILshort polypeptide, or a TRAILshort peptidomimetic described herein for administration to a mammal (e.g., a human) to reduce excessive T cell activation.

### 2. Background Information

TNF related apoptosis inducing ligand (TRAIL) is a member of the tumor necrosis factor (TNF) superfamily of death-inducing ligands whose members include Fas ligand and TNF. Ligation of TRAIL to its cognate receptors can cause cell death by apoptosis or may cause NF-κB activation (Hu et al., J. Biol. Chem., 274:30603-10 (1999)). TRAIL is widely expressed on multiple cell lineages and has potent toxicity for many tumors and virally infected cells, while sparing most healthy cells (Held et al., Drug Resist. Updat., 4:243-52 (2001); Baetu and Hiscott, Cytokine Growth Factor, 13:199-207 (2002)). TRAIL can bind to five different TRAIL receptors (TRAIL-R1, -R2, -R3, -R4, and osteoprotegerin (OPG)); cell death occurs when TRAIL binds to TRAIL-R1 or TRAIL-R2. TRAILshort is a splice variant of TRAIL that is capable of blocking TRAIL mediated cell death.

### SUMMARY

This document provides methods and materials for modulating (e.g., reducing) T cell activation (e.g., T cell proliferation, T cell effector functions, and/or cytokine secretion) using a TRAILshort polypeptide, a variant TRAILshort polypeptide, or a TRAILshort peptidomimetic described herein. For example, a TRAILshort polypeptide, a variant TRAILshort polypeptide, or a TRAILshort peptidomimetic described herein can be administered to a mammal in need thereof, e.g., a human with an autoimmune disorder, graft versus host disease, lichen planus or other disorder characterized by excessive T cell activation, to reduce excessive T cell activation (e.g., excessive T cell proliferation, T cell effector functions, and/or cytokine secretion). This document also provides compositions containing particles (e.g., microparticles and/or nanoparticles) designed to include a TRAILshort polypeptide, a variant TRAILshort polypeptide, or a TRAILshort peptidomimetic described herein for administration to a mammal (e.g., a human) to reduce excessive T cell activation.

As described herein, a TRAILshort polypeptide (or a variant TRAILshort polypeptide or a TRAILshort peptidomimetic) can be administered to a mammal to reduce T cell proliferation, T cell effector functions, and/or cytokine secretion within the mammal. Having the ability to reduce excessive T cell activation within a mammal (e.g., a human) using the methods and materials provided herein can allow clinicians and patients to treat conditions associated with excessive T cell activation such as autoimmune disease (e.g., multiple sclerosis, rheumatoid arthritis, type 1 diabetes, systemic lupus erythematosus, Addison's disease, Graves' disease, Sjogren's syndrome, Hashimoto's thyroiditis, autoimmune vasculitis, organ transplant rejection, Celiac disease, pernicious anemia, psoriatic arthritis, or psoriasis), graft versus host disease, inflammatory bowel diseases (e.g., ulcerative colitis and Crohn's disease), and lichen planus.

In a first aspect, this document features a method for reducing excessive T cell activation in a mammal in need thereof. The method can include, or consist essentially of, administering to the mammal a composition comprising a TRAILshort polypeptide, a variant TRAILshort polypeptide, or a TRAILshort fusion polypeptide, wherein T cell activation is reduced within the mammal. The mammal can be a human. The mammal can have an autoimmune disease, graft versus host disease, or lichen planus. The mammal can have been identified as having an autoimmune disease, graft versus host disease, or lichen planus prior to the administering step. The autoimmune disease can be multiple sclerosis, rheumatoid arthritis, type 1 diabetes, systemic lupus erythematosus, Addison's disease, Graves' disease, Sjogren's syndrome, Hashimoto's thyroiditis, autoimmune vasculitis, organ transplant rejection, Celiac disease, pernicious anemia, psoriatic arthritis, or psoriasis.

In another aspect, this document features a method for treating an autoimmune disease in a mammal in need thereof, where the method includes, or consists essentially of, administering to the mammal a composition comprising a TRAILshort polypeptide, a variant TRAILshort polypeptide, or a TRAILshort fusion polypeptide, wherein excessive T cell activation is reduced within the mammal, thereby treating the autoimmune disease. The TRAILshort polypeptide can include, consist of, or consist essentially of the amino acid sequence of SEQ ID NO:1. The TRAILshort polypeptide can lack a transmembrane domain. The TRAILshort polypeptide can include, consist of, or consist essentially of, the amino acid sequence of SEQ ID NO:2. The variant TRAILshort polypeptide can be polypeptide having the amino acid sequence set forth in SEQ ID NO:1, where the amino acid residue at position 43 is replaced with an arginine residue, the amino acid residue at position 81 is replaced with a histidine residue, and/or the amino acid residue at position 85 is replaced with a lysine residue. The method can include administering a TRAILshort fusion polypeptide to the mammal, where the fusion polypeptide comprises an affinity tag and a TRAILshort polypeptide or variant TRAILshort polypeptide. The affinity tag can be a polyhistidine tag or a c-myc tag. The composition can include a plurality of particles, wherein each particle of the plurality comprises the TRAILshort polypeptide, the variant TRAILshort polypeptide, or the TRAILshort fusion polypeptide. The particles can be microparticles, exosomes, nanoparticles, or extracellular vesicles. At least a portion of the TRAILshort polypeptide, the variant of a TRAILshort polypeptide, or the TRAILshort fusion polypeptide can be present on the surface of the particles.

In another aspect, this document features a method for treating graft versus host disease in a mammal in need thereof, where the method includes, or consists essentially of, administering to the mammal a composition comprising a TRAILshort polypeptide, a variant TRAILshort polypeptide, or a TRAILshort fusion polypeptide, wherein excessive T cell activation is reduced within the mammal, thereby treating the graft versus host disease. The TRAILshort polypeptide can include, consist of, or consist essentially of the amino acid sequence of SEQ ID NO:1. The TRAILshort polypeptide can lack a transmembrane domain. The TRAILshort polypeptide can include, consist of, or consist essentially of, the amino acid sequence of SEQ ID NO:2. The variant TRAILshort polypeptide can be polypeptide having the amino acid sequence set forth in SEQ ID NO:1, where the amino acid residue at position 43 is replaced with an arginine residue, the amino acid residue at position 81 is replaced with a histidine residue, and/or the amino acid residue at position 85 is replaced with a lysine residue. The method can include administering a TRAILshort fusion polypeptide to the mammal, where the fusion polypeptide comprises an affinity tag and a TRAILshort polypeptide or variant TRAILshort polypeptide. The affinity tag can be a polyhistidine tag or a c-myc tag. The composition can include a plurality of particles, wherein each particle of the plurality comprises the TRAILshort polypeptide, the variant TRAILshort polypeptide, or the TRAILshort fusion polypeptide. The particles can be microparticles, exosomes, nanoparticles, or extracellular vesicles. At least a portion of the TRAILshort polypeptide, the variant of a TRAILshort polypeptide, or the TRAILshort fusion polypeptide can be present on the surface of the particles.

In still another aspect, this document features a method for treating lichen planus in a mammal in need thereof, where the method includes, or consists essentially of, administering to the mammal a composition comprising a TRAILshort polypeptide, a variant TRAILshort polypeptide, or a TRAILshort fusion polypeptide, wherein excessive T cell activation is reduced within the mammal, thereby treating the lichen planus. The TRAILshort polypeptide can include, consist of, or consist essentially of the amino acid sequence of SEQ ID NO:1. The TRAILshort polypeptide can lack a transmembrane domain. The TRAILshort polypeptide can include, consist of, or consist essentially of, the amino acid sequence of SEQ ID NO:2. The variant TRAILshort polypeptide can be polypeptide having the amino acid sequence set forth in SEQ ID NO:1, where the amino acid residue at position 43 is replaced with an arginine residue, the amino acid residue at position 81 is replaced with a histidine residue, and/or the amino acid residue at position 85 is replaced with a lysine residue. The method can include administering a TRAILshort fusion polypeptide to the mammal, where the fusion polypeptide comprises an affinity tag and a TRAILshort polypeptide or variant TRAILshort polypeptide. The affinity tag can be a polyhistidine tag or a c-myc tag. The composition can include a plurality of particles, wherein each particle of the plurality comprises the TRAILshort polypeptide, the variant TRAILshort polypeptide, or the TRAILshort fusion polypeptide. The particles can be microparticles, exosomes, nanoparticles, or extracellular vesicles. At least a portion of the TRAILshort polypeptide, the variant of a TRAILshort polypeptide, or the TRAILshort fusion polypeptide can be present on the surface of the particles.

In another aspect, this document features a pharmaceutical composition comprising a plurality of particles, wherein each particle of the plurality comprises a TRAILshort polypeptide, a variant TRAILshort polypeptide, or a TRAILshort fusion polypeptide. At least a portion of the TRAILshort polypeptide, the variant of a TRAILshort polypeptide, or the TRAILshort fusion polypeptide can be present on the surface of the particles. The TRAILshort polypeptide can include, consist of, or consist essentially of the amino acid sequence of SEQ ID NO:1. The TRAILshort polypeptide can lack a transmembrane domain. The TRAILshort polypeptide can include, consist of, or consist essentially of, the amino acid sequence of SEQ ID NO:2. The variant TRAILshort polypeptide can include a polypeptide having the amino acid sequence set forth in SEQ ID NO:1 where the amino acid residue at position 43 is replaced with an arginine residue, the amino acid residue at position 81 is replaced with a histidine residue, and/or the amino acid residue at position 85 is replaced with a lysine residue. The composition can further include an anti-inflammatory agent. The anti-inflammatory agent can be selected from the group consisting of inhibitors of Receptor Interacting Serine/Threonine Kinase 1 (Ripk1), inhibitors of tumor necrosis factor (TNF), inhibitors of interleukin 6 (IL6), inhibitors of IL6 receptor (IL6R), inhibitors of interleukin 1 beta (IL-1b), inhibitors of platelet-activating factor (PAF), inhibitors of CD40 ligand (CD40L), inhibitors of interleukin 4 receptor (IL4R), inhibitors of Burton tyrosine kinase (BTK), inhibitors of TNF Superfamily member 4 (OX40L), inhibitors of Complement, inhibitors of interleukin 23 (IL23), inhibitors of interleukin 13 (IL13), inhibitors of interleukin 2 (IL2), and inhibitors of Rho Associated Coiled-Coil Containing Protein Kinase 2 (ROCK2). The composition can further include an immunosuppressant. The immunosuppressant can be selected from the group consisting of cyclosporin, tacrolimus, sirolimus, everolimus, mycophenolate, steroids, hydroxychloroquine, cyclophosphamides, Jak inhibitors, Stat inhibitors, azathioprine, OKT3, basiliximab, daclizumab, abatacept, adalimumab, anakinra, certolizumab, etanercept, golimumab, infliximab, ixekizumab, natalizumab, rituximab, secukinumab, tocilizumab, ustekinumab, vedolizumab, basiliximab, and daclizumab.

Unless otherwise defined, all technical and scientific terms used herein have the same meaning as commonly understood by one of ordinary skill in the art to which this disclosure pertains. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A-1C: TRAILshort (Ts) or extracellular vesicles containing TRAILshort (TsEV) reduce phosphorylation induced by T cell receptor (TCR) signaling. FIG. 1A is an image showing a western blot for TCR-associated signaling molecules. CD3+ T cells were isolated from uninfected donor(s) and rested overnight in RPMI media containing 1% fetal bovine serum (FBS) and TL2 (20 IU/mL). Rested cells were added to plates coated with TRAILshort peptide (101 amino acids, SEQ ID NO:1; 5 μg/mL) or BSA for one hour, followed by stimulation with CD3/CD28 beads (1 bead:2 cells) for 30 minutes and then western blot analysis to detect TCR-associated signaling molecules. Pretreatment of cells with the Ts peptide resulted in less ZAP70 phosphorylated at position 319, less LAT phosphorylated at position 191, less Syk phosphorylated at position 535/536, and less PLC gamma phosphorylated at position 783, as compared to treatment with CD3/CD28 alone or pretreatment with BSA. FIG. 1B is an image showing another western blot to detect TCR-associated signaling molecules. For this study, after incubation with RPMI media containing 1% serum and IL2 overnight, cells were treated with extracellular vesicles containing TRAILshort (TsEVs), extracellular vesicles containing TRAILshort lacking the transmembrane domain (TsΔTmEVs), or empty extracellular vesicles (Empty EVs) (1,000 EVs/cell) for three hours, followed by western blot analysis to detect TCR-associated signaling molecules. Pretreatment of cells with TsEV resulted in less phosphorylation of ZAP70 at position 319, less phosphorylation of LAT at position 220, and less phosphorylation of SLP76 at position 376, as compared to CD3/CD28 treatment alone or pretreatment with TsΔTmEV. FIG. 1C includes a pair of histograms plotting data from studies in which a group of enriched CD3 positive cells (containing CD4+ and CD8+ T cells) were simultaneously stimulated with CD3/CD28 beads and treated with either TsEVs or TsΔTmEVs for 24 hours, followed by p-ZAP70 (Y319) detection by flow cytometry in the enriched CD4 and CD8 cell populations. The “unstained” and “isotype” control samples utilized CD3 cells that contained a mixture of CD4 and CD8 cells. These studies demonstrated that the level of phosphorylated ZAP70 (Y319) was reduced in cells treated with TsEV.

FIG. 2 is a schematic showing the workflow for the ELISpot experiments and analysis.

FIG. 3A includes graphs plotting the results of ELISpot assays performed in the presence of TRAILshort peptide (Ts) vs. BSA control (top), or TsEV vs. TsΔTmEV (bottom) in uninfected healthy donors. Cells were treated with 0.1% BSA alone or with added Ts peptide (5 μg/mL), TsEV (cell:EV=1:1000), or TsΔTmEV (cell:EV=1:1000), and stimulated with CD3/CD28 beads (bead:cell=1:2) for 24 hours. The beads were removed, and the cells were stimulated with recall antigens (tetanus toxoid for uninfected cells) for an additional 24 hours, followed by IFNγ ELISpot assay. Uninfected donor CD3+ cells: Ts peptide, n=25; TsEV, n=22. Uninfected donor CD8+ cells: Ts peptide, n=11; TsEV, n=23. FIG. 3B includes a pair of graphs plotting the results of ELISpot assays performed with cells from HIV infected donors. Cells were treated with 0.1% BSA alone or with added Ts peptide (5 μg/ml) (left), or TsEV (cell:EV=1:1000) or TsΔTmEV (cell:EV=1:1000) (right) and stimulated with CD3/CD28 beads (bead:cell=1:2) for 24 hours. The beads were removed, and the cells were stimulated with recall antigens included an HIV peptide mixture (a combination of HIV-1 gag/pol and nef antigens) for an additional 24 hours, followed by IFNγ ELISpot assay. HIV infected CD3+ cells: Ts peptide vs. BSA, n=15; TsEV vs. TsΔTm, n=12. P<0.05 indicates significant values. These studies demonstrated that Ts peptide and TsEV reduced ELISpot in cells from both HIV negative donors and HIV positive donors.

FIG. 4 includes a pair of graphs plotting ELISpot responses in T cells from lymphoma patients stimulated with autologous lymphoma cells in the presence or absence of TRAILshort antibody. CD3+ T cells isolated from lymphoma patients were treated with isotype or Ts Ab (5 μg/mL). Cells were then stimulated with CD3/28 beads (bead:cell=1:2) for 24 hours. The beads were removed, and the cells were stimulated with autologous, malignant CD19+ tumor B-cells for an additional 24 hours, followed by IFNγ ELISpot assay. The data plotted in the graphs are from two different patients. Marginal Zone Lymphoma (n=3 replicates); Diffuse Large B-cell Lymphoma (n=3 replicates). P<0.05 indicates significant values. These studies demonstrated that the TRAILshort Ab enhanced the ELISpot responses.

FIG. 5 is an image showing a western blot. TRAILshort extracellular vesicles (TsEV) reduced the interaction of phosphorylated ZAP70 (Y319) with the CD3 zeta chain following CD3/CD28 activation. Uninfected primary CD3+ T cells were cultured in full RPMI media containing 50U IL-2 overnight. Cells were treated with either TsΔTmEV or TsEV (1:1000=Cell: EV), in the presence or absence of CD3/CD28 beads (1:1=bead:cell) for 30 minutes. Cells were then collected, and the lysate was subject to anti-CD3zeta immunoprecipitation (IP). The immunoprecipitates were used to detect the co-immunoprecipitated proteins/phospho proteins by western blot, including total and phosphorylated ZAP70 (Y319) using the corresponding antibodies. In parallel, whole cell lysates were blotted for the same proteins. These studies demonstrated that TsEV reduced the amount of phosphorylated ZAP70 (Y319) that was associated with CD3 zeta.

### DETAILED DESCRIPTION

This document provides methods and materials for reducing excessive T cell activation in a mammal in need thereof (e.g., a mammal having an autoimmune disease, graft versus host disease, or lichen planus) using compositions that include TRAILshort polypeptides, variants of TRAILshort polypeptides, TRAILshort peptidomimetics, or fusion polypeptides containing a TRAILshort polypeptide fused to one or more other polypeptides (e.g., an IgG4 polypeptide). Any appropriate mammal can be treated as described herein including, without limitation, humans, monkeys, dogs, horses, sheep, pigs, goats, rabbits, rats, or mice. In some cases, the methods described herein can be used for reducing excessive T cell activation in a mammal identified as having an autoimmune disease (e.g., multiple sclerosis, rheumatoid arthritis, type 1 diabetes, systemic lupus erythematosus, Addison's disease, Graves' disease, Sjogren's syndrome, Hashimoto's thyroiditis, autoimmune vasculitis, organ transplant rejection, Celiac disease, pernicious anemia, psoriatic arthritis, or psoriasis), graft versus host disease, or lichen planus.

A non-limiting example of a TRAILshort polypeptide that can be used as described herein is set forth in SEQ ID NO:1:

(full length TRAILshort; SEQ ID NO: 1)
MAMMEVQGGPSLGQTCVLIVIFTVLLQSLCVAVTYVYFTNELKQM
QDKYSKSGIACFLKEDDSYWDPNDEESMNSPCWQVKWQLRQLVR
KTPRMKRLWAAK

The first 90 amino acids of SEQ ID NO:1 are the same as the first 90 amino acids of a full-length TRAIL polypeptide (see, e.g., NCBI Reference Sequence No. NP_001177871), while the last 11 amino acids of the C-terminus are distinct (TPRMKRLWAAK; SEQ ID NO:2). A representative nucleic acid sequence encoding the TS polypeptide having the amino acid sequence of SEQ ID NO:1 is set forth in SEQ ID NO:3:

(SEQ ID NO: 3)
ATGGCTATGATGGAGGTCCAGGGGGGACCCAGCCTGGGACAGA
CCTGCGTGCTGATCGTGATCTTCACAGTGCTCCTGCAGTCTCTCT
GTGTGGCTGTAACTTACGTGTACTTTACCAACGAGCTGAAGCAG
ATGCAGGACAAGTACTCCAAAAGTGGCATTGCTTGTTTCTTAAA
AGAAGATGACAGTTATTGGGACCCCAATGACGAAGAGAGTATGA
ACAGCCCCTGCTGGCAAGTCAAGTGGCAACTCCGTCAGCTCGTT
AGAAAGACTCCAAGAATGAAAAGGCTCTGGGCCGCAAAATAA

Another non-limiting example of a TRAILshort polypeptide that can be used as described herein is set forth in SEQ ID NO:4:

(TRAILshort aa 39-101; SEQ ID NO: 4)
TNELKQMQDKYSKSGIACFLKEDDSYWDPNDEESMNSPCWQVK
WQLRQLVRKTPRMKRLWAAK

In some cases, a TRAILshort polypeptide comprises, consists essentially of, or consists of the amino acid sequence set forth in SEQ ID NO:1.

In some cases, a variant TRAILshort polypeptide can have at least 70 percent sequence identity to SEQ ID NO:1. For example, a variant TRAILshort polypeptide can have at least 75, 80, 85, 90, 95, 98, or 99 percent identity to SEQ ID NO:1, provided that it includes at least one amino acid substitution compared to SEQ ID NO:1.

The percent identity between a particular amino acid sequence and the amino acid sequence set forth in SEQ ID NO:1 can be determined as follows. First, the amino acid sequences are aligned using the BLAST 2 Sequences (Bl2seq) program from the stand-alone version of BLASTZ containing BLASTP version 2.0.14. This stand-alone version of BLASTZ can be obtained from Fish & Richardson's web site (e.g., www.fr.com/blast/) or the State University of New York-Old Westbury Library (call number: QH 447.M6714). Instructions explaining how to use the Bl2seq program can be found in the readme file accompanying BLASTZ. Bl2seq performs a comparison between two amino acid sequences using the BLASTP algorithm. To compare two amino acid sequences, the options of Bl2seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\Bl2seq - ic:\seq1.txt- j c:seq2.txt -p blastp -o c:\output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical amino acid residue is presented in both sequences. The percent identity is determined by dividing the number of matches by the length of the full-length amino acid sequence set forth in SEQ ID NO:1 followed by multiplying the resulting value by 100. For example, an amino acid sequence that has 98 matches when aligned with the sequence set forth in SEQ ID NO:1 is 97.0 percent identical to the sequence set forth in SEQ ID NO:1 (i.e., 98+101*100=97.0).

It is noted that the percent identity value is rounded to the nearest tenth. For example, 78.11, 78.12, 78.13, and 78.14 are rounded down to 78.1, while 78.15, 78.16, 78.17, 78.18, and 78.19 are rounded up to 78.2. It also is noted that the length value will always be an integer.

In some cases, a variant TRAILshort polypeptide can have from one to 20 (e.g., 1, 2, 3, 4, 5, 6, 7, 8, 9, 10, 11, 12, 13, 14, 15, 16, 17, 18, 19, or 20) amino acid modifications within SEQ ID NO:1 provided the variant TRAILshort polypeptide is capable of inhibiting T cell activation. Such amino acid modifications can include, without limitation, amino acid substitutions, amino acid deletions, amino acid additions, and combinations thereof. In some cases, an amino acid modification can be made to improve the binding and/or to improve a functional activity of a TRAILshort polypeptide provided herein. In some cases, an amino acid substitution within an articulated sequence identifier can be a conservative amino acid substitution. For example, conservative amino acid substitutions can be made by substituting one amino acid residue for another amino acid residue having a similar side chain. Families of amino acid residues having similar side chains can include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine).

In some cases, an amino acid substitution within an articulated sequence identifier can be a non-conservative amino acid substitution. Non-conservative amino acid substitutions can be made by substituting one amino acid residue for another amino acid residue having a dissimilar side chain. Examples of non-conservative substitutions include, without limitation, substituting (a) a hydrophilic residue (e.g., serine or threonine) for a hydrophobic residue (e.g., leucine, isoleucine, phenylalanine, valine, or alanine); (b) a cysteine or proline for any other residue; (c) a residue having a basic side chain (e.g., lysine, arginine, or histidine) for a residue having an acidic side chain (e.g., aspartic acid or glutamic acid); and (d) a residue having a bulky side chain (e.g., phenylalanine) for glycine or other residue having a small side chain.

Examples of variant TRAILshort polypeptides that can be used as described herein include, without limitation, those set forth in TABLE 1. In some cases, a variant TRAILshort polypeptide can have the amino acid sequence set forth in SEQ ID NO:1 where the amino acid residue at position 43 is replaced with an arginine residue, the amino acid residue at position 81 is replaced with a histidine residue, and/or the amino acid residue at position 85 is replaced with a lysine residue. For example, a variant TRAILshort polypeptide can have the amino acid sequence set forth in SEQ ID NO:1 wherein the amino acid residue at position 43 is replaced with an arginine residue and the amino acid residue at position 85 is replaced with a lysine residue. For example, a variant TRAILshort polypeptide can have the amino acid sequence set forth in SEQ ID NO:1 wherein the amino acid residue at position 43 is replaced with an arginine residue and the amino acid residue at position 81 is replaced with a histidine residue. For example, a variant TRAILshort polypeptide can have the amino acid sequence set forth in SEQ ID NO:1 wherein the amino acid residue at position 81 is replaced with a histidine residue and the amino acid residue at position 85 is replaced with a lysine residue.

TABLE 1

| | SEQ |
|---|---|
| | ID |
| Variants of TRAILshort polypeptide | NO: |
| MAMMEVQGGPSLGQTCVLIVIFTVLLQSLCVAVTYVYFTNELRQM | 5 |
| QDKYSKSGIACFLKEDDSYWDPNDEESMNSPCWQVKWQLRQLVR | |
| KTPRMKRLWAAK (SEQ ID NO: 1 with Arg at position 43) | |
| MAMMEVQGGPSLGQTCVLIVIFTVLLQSLCVAVTYVYFTNELKQM | 6 |
| QDKYSKSGIACFLKEDDSYWDPNDEESMNSPCWQVKWQLKQLVR | |
| KTPRMKRLWAAK (SEQ ID NO: 1 with Lys at position 85) | |
| MAMMEVQGGPSLGQTCVLIVIFTVLLQSLCVAVTYVYFTNELKQM | 7 |
| QDKYSKSGIACFLKEDDSYWDPNDEESMNSPCWQVHWQLRQLVR | |
| KTPRMKRLWAAK (SEQ ID NO: 1 with His at position 81) | |
| MAMMEVQGGPSLGQTCVLIVIFTVLLQSLCVAVTYVYFTNELKQM | 8 |
| QDKYSKSGIACFLKEDDSYWDPNDEESMNSPCWQVHWQLKQLVR | |
| KTPRMKRLWAAK (SEQ ID NO: 1 with His at position 81 and Lys at | |
| position 85) | |
| MAMMEVQGGPSLGQTCVLIVIFTVLLQSLCVAVTYVYFTNELRQM | 9 |
| QDKYSKSGIACFLKEDDSYWDPNDEESMNSPCWQVHWQLKQLVR | |
| KTPRMKRLWAAK (SEQ ID NO: 1 with Arg at position 43, His at position | |
| 81, and Lys at position 85) | |
| MAMMEVQGGPSLGQTCVLIVIFTVLLQSLCVAVTYVYFTNELRQM | 10 |
| QDKYSKSGIACFLKEDDSYWDPNDEESMNSPCWQVKWQLKQLVR | |
| KTPRMKRLWAAK (SEQ ID NO: 1 with Arg at position 43 and Lys at | |
| position 85) | |
| MAMMEVQGGPSLGQTCVLIVIFTVLLQSLCVAVTYVYFTNELRQM | 11 |
| QDKYSKSGIACFLKEDDSYWDPNDEESMNSPCWQVHWQLRQLVR | |
| KTPRMKRLWAAK (SEQ ID NO: 1 with Arg at position 43 and His at | |
| position 81) | |
| TNELRQMQDKYSKSGIACFLKEDDSYWDPNDEESMNSPCWQVK | 12 |
| WQLRQLVRKTPRMKRLWAAK (SEQ ID NO: 2 with Arg at position 5) | |
| TNELKQMQDKYSKSGIACFLKEDDSYWDPNDEESMNSPCWQVK | 13 |
| WQLKQLVRKTPRMKRLWAAK (SEQ ID NO: 2 with Lys at position 47) | |
| TNELKQMQDKYSKSGIACFLKEDDSYWDPNDEESMNSPCWQVH | 14 |
| WQLRQLVRKTPRMKRLWAAK (SEQ ID NO: 2 with His at position 43) | |
| TNELKQMQDKYSKSGIACFLKEDDSYWDPNDEESMNSPCWQVH | 15 |
| WQLKQLVRKTPRMKRLWAAK (SEQ ID NO: 2 with His at position 43 | |
| and Lys at position 47) | |
| TNELRQMQDKYSKSGIACFLKEDDSYWDPNDEESMNSPCWQVH | 16 |
| WQLKQLVRKTPRMKRLWAAK (SEQ ID NO: 2 with Arg at position 5, | |
| His at position 43, and Lys at position 47) | |
| TNELRQMQDKYSKSGIACFLKEDDSYWDPNDEESMNSPCWQVK | 17 |
| WQLKQLVRKTPRMKRLWAAK (SEQ ID NO: 2 with Arg at position 5 | |
| and Lys at position 47) | |
| TNELRQMQDKYSKSGIACFLKEDDSYWDPNDEESMNSPCWQVH | 18 |
| WQLRQLVRKTPRMKRLWAAK (SEQ ID NO: 2 with Arg at position 5 | |
| and His at position 43) | |

Methods for generating an amino acid sequence variant (e.g., an amino acid sequence that includes one or more modifications with respect to an articulated sequence identifier) can include site-specific mutagenesis or random mutagenesis (e.g., by PCR) of a nucleic acid encoding the polypeptide. See, for example, Zoller, Curr. Opin. Biotechnol. 3: 348-354 (1992). Both naturally occurring and non-naturally occurring amino acids (e.g., artificially-derivatized amino acids) can be used to generate an amino acid sequence variant provided herein.

In some cases, this document provides TRAILshort peptidomimetics. A TRAILshort peptidomimetic can be protease resistant and/or can have an increased biological half-life. In some cases, a TRAILshort peptidomimetic can have a backbone that is partially or completely non-peptidic, but with side groups identical to the side groups of the amino acid residues that occur in the polypeptide on which the peptidomimetic is based (e.g., SEQ ID NO:1). In some cases, a peptidomimetic can be resistant to proteases and can have a non-peptide backbone, including, e.g., ester, thioester, thioamide, retroamide, reduced carbonyl, dimethylene, or ketomethylene bonds. In some cases, a peptidomimetic provided herein can contain chemical structures such as ε-aminohexanoic acid; hydroxylated amino acids such as 3-hydroxyproline, 4-hydroxyproline, (5R)-5-hydroxy-L-lysine, allo-hydroxylysine, and 5-hydroxy-L-norvaline; or glycosylated amino acids such as amino acids containing monosaccharides (e.g., D-glucose, D-galactose, D-mannose, D-glucosamine, and D-galactosamine) or combinations of monosaccharides.

In some cases, a TRAILshort polypeptide, variant TRAILshort polypeptide, or peptidomimetic provided herein can be fused or conjugated (e.g., covalently or non-covalently attached) to another polypeptide or other moiety to provide a fusion protein or conjugate. For example, a TRAILshort polypeptide, variant TRAILshort polypeptide, or peptidomimetic provided herein can be conjugated (e.g., covalently or non-covalently attached) to a polymer (e.g., polyethylene glycol (PEG), polyethylenimine (PEI) modified with PEG (PEI-PEG), and/or polyglutamic acid (PGA) (N-(2-Hydroxypropyl) methacrylamide (HPMA) copolymers), hyaluronic acid, a fluorescent substance, a luminescent substance, a hapten, an affinity tag (e.g., c-myc, hemagglutinin, polyhistidine, or Flag™ (Kodak)), an enzyme (e.g., an enzyme that aids in the detection of a polypeptide, such as alkaline phosphatase), a metal chelate, a drug, a radioisotope, and/or a cytotoxic agent. Any appropriate method can be used to conjugate (e.g., covalently or non-covalently attach) another polypeptide or other moiety to a TRAILshort polypeptide, variant TRAILshort polypeptide, or peptidomimetic provided herein provided herein. For example, another polypeptide or other moiety can be conjugated to a TRAILshort polypeptide, variant TRAILshort polypeptide, or peptidomimetic provided herein using the methods described in U.S. Pat. No. 8,021,661. Examples of TRAILshort fusion polypeptides are provided in TABLE 2.

TABLE 2

| | SEQ ID |
|---|---|
| TRAILshort fusion polypeptide* | NO: |
| MINELKQMQDKYSKSGIACFLKEDDSYWDPNDEESMNSPCWQ | 19 |
| VKWQLRQLVRKTPRMKRLWAAKYSSTMVRSYGPPCPPCPAPEF | |
| LGGPSVFLFPPKPKDTLMISRTPEVTCVVVDVSQEDPEVQFNWY | |
| VDGVEVHNAKTKPREEQFNSTYRVVSVLTVLHQDWLNGKEYK | |
| CKVSNKGLPSSIEKTISKAKGQPREPQVYTLPPSQEEMTKNQVSL | |
| TCLVKGFYPSDIAVEWESNGQPENNYKTTPPVLDSDGSFFLYSR | |
| LTVDKSRWQEGNVFSCSVMHEALHNHYTQKSLSLSPGK | |
| (TRAILshort aa 39-101-IgG4-FC-C-terminus) | |
| MYSSTMVRSYGPPCPPCPAPEFLGGPSVFLFPPKPKDTLMISRTPE | 20 |
| VTCVVVDVSQEDPEVQFNWYVDGVEVHNAKTKPREEQFNSTYR | |
| VVSVLTVLHQDWLNGKEYKCKVSNKGLPSSIEKTISKAKGQPRE | |
| PQVYTLPPSQEEMTKNQVSLTCLVKGFYPSDIAVEWESNGQPEN | |
| NYKTTPPVLDSDGSFFLYSRLTVDKSRWQEGNVFSCSVMHEALH | |
| NHYTQKSLSLSPGKTNELKQMQDKYSKSGIACFLKEDDSYWDPN | |
| DEESMNSPCWQVKWQLRQLVRKTPRMKRLWAAK (TRAILshort aa 39- | |
| 101-IgG4-FC-N-terminus) | |
| MAMMEVQGGPSLGQTCVLIVIFTVLLQSLCVAVTYVYFTNELKQ | 21 |
| MQDKYSKSGIACFLKEDDSYWDPNDEESMNSPCWQVKWQLRQL | |
| VRKTPRMKRLWAAKMYSSTMVRSYGPPCPPCPAPEFLGGPSVFL | |
| FPPKPKDTLMISRTPEVTCVVVDVSQEDPEVQFNWYVDGVEVHN | |
| AKTKPREEQFNSTYRVVSVLTVLHQDWLNGKEYKCKVSNKGLP | |
| SSIEKTISKAKGQPREPQVYTLPPSQEEMTKNQVSLTCLVKGFYPS | |
| DIAVEWESNGQPENNYKTTPPVLDSDGSFFLYSRLTVDKSRWQE | |
| GNVFSCSVMHEALHNHYTQKSLSLSPGK (TRAILshort full-length- | |
| human IgG4-FC-C-terminus) | |
| MYSSTMVRSYGPPCPPCPAPEFLGGPSVFLFPPKPKDTLMISRTPE | 22 |
| VTCVVVDVSQEDPEVQFNWYVDGVEVHNAKTKPREEQFNSTYR | |
| VVSVLTVLHQDWLNGKEYKCKVSNKGLPSSIEKTISKAKGQPRE | |
| PQVYTLPPSQEEMTKNQVSLTCLVKGFYPSDIAVEWESNGQPEN | |
| NYKTTPPVLDSDGSFFLYSRLTVDKSRWQEGNVFSCSVMHEAL | |
| HNHYTQKSLSLSPGKMAMMEVQGGPSLGQTCVLIVIFTVLLQSL | |
| CVAVTYVYFTNELKQMQDKYSKSGIACFLKEDDSYWDPNDEES | |
| MNSPCWQVKWQLRQLVRKTPRMKRLWAAK (TRAILshort full-length- | |
| human IgG4-Fc-N-terminus) | |
| TNELKQMQDKYSKSGIACFLKEDDSYWDPNDEESMNSPCWQVK | 23 |
| WQLRQLVRKTPRMKRLWAAKMYSSTMVRSYGPPCPPCPAPEFL | |
| GGPSVFLFPPKPKDTLMISRTPEVTCVVVDVSQEDPEVQFNWYVD | |
| GVEVHNAKTKPREEQFNSTYRVVSVLTVLHQDWLNGKEYKCKV | |
| SNKGLPSSIEKTISKAKGQPREPQVYTLPPSQEEMTKNQVSLTCLV | |
| KGFYPSDIAVEWESNGQPENNYKTTPPVLDSDGSFFLYSRLTVDK | |
| SRWQEGNVFSCSVMHEALHNHYTQKSLSLSPGK (TRAIL short aa | |
| 39-101-human IgG4-FC) | |
| MAMMEVQGGPSLGQTCVLIVIFTVLLQSLCVAVTYVYFTNELKQ | 24 |
| MQDKYSKSGIACFLKEDDSYWDPNDEESMNSPCWQVKWQLRQL | |
| VRKTPRMKRLWAAKMTNKCLLQIALLLCFSTTALSMSYNLLGFL | |
| QRSSNFQCQKLLWQLNGRLEYCLKDRMNFDIPEEIKQLQQFQKE | |
| DAALTIYEMLQNIFAIFRQDSSSTGWNETIVENLLANVYHQINHLK | |
| TVLEEKLEKEDFTRGKLMSSLHLKRYYGRILHYLKAKEYSHCAW | |
| TIVR VEILRNFYFINRLTGYLRN (TRAIL short full-length-interferon | |
| beta) | |
| MTNKCLLQIALLLCFSTTALSMSYNLLGFLQRSSNFQCQKLLW | 25 |
| QLNGRLEYCLKDRMNFDIPEEIKQLQQFQKEDAALTIYEMLQN | |
| IF AIFRQDSSSTGWNETIVENLLANVYHQINHLKTVLEEKLEKE | |
| DFTRGKLMSSLHLKRYYGRILHYLKAKEYSHCAWTIVRVEILR | |
| NFYFINRLTGYLRNMAMMEVQGGPSLGQTCVLIVIFTVLLQSL | |
| CVAVTYVYFTNELKQMDKYSKSGIACFLKEDDSYWDPNDEES | |
| MNSPCWQVKWQLRQLVRKTPRMKRLWAAK (interferon beta- | |
| TRAILshort full-length) | |
| TNELKQMQDKYSKSGIACFLKEDDSYWDPNDEESMNSPCWQVK | 26 |
| WQLRQLVRKTPRMKRLWAAKMTNKCLLQIALLLCFSTTALSMS | |
| YNLLGFLQRSSNFQCQKLLWQLNGRLEYCLKDRMNFDIPEEIKQ | |
| LQQFQKEDAALTIYEMLQNIFAIFRQDSSSTGWNETIVENLLANV | |
| YHQINHLKTVLEEKLEKEDFTRGKLMSSLHLKRYYGRILHYLKA | |
| KEYSHCAWTIVRVEILRNFYFINRLTGYLRN (TRAILshort aa 39-101- | |
| interferon beta) | |
| MTNKCLLQIALLLCFSTTALSMSYNLLGFLQRSSNFQCQKLLW | 27 |
| QLNGRLEYCLKDRMNFDIPEEIKQLQQFQKEDAALTIYEMLQN | |
| IFAIFRQDSSSTGWNETIVENLLANVYHQINHLKTVLEEKLEKE | |
| DFTRGKLMSSLHLKRYYGRILHYLKAKEYSHCAWTIVRVEILR | |
| NFYFINRLTGYLRNTNELKQMQDKYSKSGIACFLKEDDSYW | |
| DPNDEESMNSPCWQVKWQLRQLVRKTPRMKRLWAAK (interferon | |
| beta-TRAILshort aa 39-101) | |

*Italicized sequences indicate the TRAILshort portion of each fusion polypeptide.

In some cases, a TRAILshort polypeptide (e.g., a polypeptide having the sequence set forth in SEQ ID NO:1 or SEQ ID NO:4) or a variant thereof can be fused to an antibody (e.g., a human anti-CD3 antibody, a human anti-CD4 antibody, a human anti-CD14 antibody, a human anti-CD56 antibody, a human anti-PD-1 antibody, a human anti-PD-L1 antibody, a human anti-type 1 interferon antibody, a human anti-IL-2 antibody, a human anti-a4b7 antibody, or a human anti-CCR7 antibody, a human anti-IL-1 antibody, a human anti-TNF antibody, a human anti-IL-6 antibody, a human anti-IL-17 antibody, a human anti-IL-5 antibody, a human anti-IL-23 antibody, a human anti-IL-12 antibody, a human anti-complement antibody, a human anti-CD20 antibody, a human anti-Baff antibody, a human anti-Blys antibody, a human anti-CTLA-4 antibody, a human anti-integrin antibody). The antibody can be N-terminal or C-terminal to the TRAILshort polypeptide in the fusion polypeptide. In some cases, a TRAILshort polypeptide (e.g., a polypeptide having the sequence set forth in SEQ ID NO:1 or SEQ ID NO:4) or a variant thereof can be coupled to another molecule (e.g., cyclosporin, cyclophosphamide, tacrolimus, methotrexate, fluorouracil, mercaptopurine, hydroxychloroquine, Janus kinase inhibitors such as ruxolitinib, upadacitinib, baricitinib, tofacitinib, or abrocitinib, sirolimus, everolimus, azathioprine, mycophenolate mofetil, or mycophenolate sodium). The other molecule (e.g., cyclosporin, cyclophosphamide, tacrolimus, methotrexate, fluorouracil, mercaptopurine, hydroxychloroquine, Janus kinase inhibitors [which can include ruxolitinib, upadacitinib, baricitinib, tofacitinib, or abrocitinib], sirolimus, everolimus, azathioprine, mycophenolate mofetil, or mycophenolate sodium) can be coupled to the N-terminus or the C-terminus of the TRAILshort polypeptide. The other molecule can be linked to the TRAILshort polypeptide by direct conjugation or via a linker (e.g., a polyethylene glycol linker or an alkyl linker).

In some cases, a TRAILshort polypeptide, variant TRAILshort polypeptide, or peptidomimetic provided herein provided herein can be modified with a moiety that improves its stabilization and/or retention in circulation, for example, in blood, serum, or other tissues by, for example, at least 1.5-, 2-, 5-, 10-, or 50-fold. For example, a TRAILshort polypeptide, variant TRAILshort polypeptide, or peptidomimetic provided herein provided herein can be attached (e.g., covalently or non-covalently attached) to a polymer such as a substantially non-antigenic polymer. Examples of substantially non-antigenic polymers that can be used as described herein include, without limitation, polyalkylene oxides and polyethylene oxides. In some cases, a polymer used herein can have any appropriate molecule weight. For example, a polymer having an average molecular weight from about 200 Daltons to about 35,000 Daltons (e.g., from about 1,000 to about 15,000 Daltons or from about 2,000 to about 12,500 Daltons) can be used. In some cases, a TRAILshort polypeptide, variant TRAILshort polypeptide, or peptidomimetic provided herein can be attached (e.g., covalently or non-covalently) to a water soluble polymer. Examples of water soluble polymers that can be used as described herein include, without limitation, hydrophilic polyvinyl polymers, polyvinylalcohol, polyvinylpyrrolidone, polyalkylene oxide homopolymers, polyethylene glycol (PEG), polypropylene glycols, polyoxyethylenated polyols, and copolymers thereof and/or block copolymers thereof provided that the water solubility of the copolymer or block copolymers is maintained.

In some cases, a TRAILshort polypeptide, variant TRAILshort polypeptide, or TRAILshort peptidomimetic provided herein can be covalently attached to oligomers, such as short, amphiphilic oligomers that enable oral administration or improve the pharmacokinetic or pharmacodynamic profile of the conjugated polypeptide. The oligomers can comprise water soluble polyethylene glycol (PEG) and lipid soluble alkyls (short chain fatty acid polymers). See, for example, International Patent Application Publication No. WO 2004/047871.

In some cases, a TRAILshort polypeptide, variant TRAILshort polypeptide, or peptidomimetic provided herein can be attached (e.g., covalently or non-covalently attached) to one or more polyoxyalkylenes (e.g., polyoxyethylene, polyoxypropylene, or block copolymers of polyoxyethylene and polyoxypropylene), polymethacrylates, carbomers, branched or unbranched polysaccharides, or combinations thereof. For example, a TRAILshort polypeptide, variant TRAILshort polypeptide, or peptidomimetic provided herein provided herein can be covalently attached to polyoxyethylene.

In some cases, a TRAILshort polypeptide, variant TRAILshort polypeptide, or TRAILshort peptidomimetic provided herein can be fused to the Fc domain of an immunoglobulin molecule (e.g., an IgG1 molecule) such that active transport of the fusion polypeptide across epithelial cell barriers via the Fc receptor occurs.

This document also provides nucleic acid molecules (e.g., isolated nucleic acid molecules) having a nucleic acid sequence encoding a TRAILshort polypeptide or variant TRAILshort polypeptide provided herein. For example, an isolated nucleic acid molecule provided herein can include a nucleic acid sequence encoding a TRAILshort polypeptide. A nucleic acid provided herein (e.g., an isolated nucleic acid molecule) can be single stranded or double stranded nucleic acid of any appropriate type (e.g., DNA, RNA, or DNA/RNA hybrids).

This document also provides vectors (e.g., plasmid vectors or viral vectors) containing one or more nucleic acids provided herein. An example of a plasmid vector that can be designed to include one or more nucleic acids having a nucleic acid sequence encoding a TRAILshort polypeptide or variant TRAILshort polypeptide provided herein includes, without limitation, plasmids, phagemids and viral vectors. Examples of viral vectors include, without limitation, retroviral vectors, parvovirus-based vectors (e.g., adenoviral-based vectors and adeno-associated virus (AAV)-based vectors), lentiviral vectors (e.g., herpes simplex (HSV)-based vectors), poxviral vectors (e.g., vaccinia virus-based vectors and fowlpox virus-based vectors), and hybrid or chimeric viral vectors. For example, a viral vector having an adenoviral backbone with lentiviral components such as those described elsewhere (Zheng et al., Nat. Biotech., 18(2): 176-80 (2000); WO 98/22143; WO 98/46778; and WO 00/17376) or viral vectors having an adenoviral backbone with AAV components such as those described elsewhere (Fisher et al., Hum. Gene Ther., 7:2079-2087 (1996)) can be designed to include one or more nucleic acids having a nucleic acid sequence encoding a TRAILshort polypeptide or variant TRAILshort polypeptide provided herein.

A vector provided herein (e.g., a plasmid vector or viral vector provided herein) can include any appropriate promoter and other regulatory sequence (e.g., transcription and translation initiation and termination codons) operably linked the nucleic acid sequence encoding a TRAILshort polypeptide or variant TRAILshort polypeptide provided herein. In some cases, a promoter used to drive expression can be a constitutive promotor or a regulatable promotor. Examples of regulatable promoters that can be used as described herein include, without limitation, inducible promotors, repressible promotors, and tissue-specific promoters. Examples of viral promotors that can be used as described herein include, without limitation, adenoviral promotors, vaccinia virus promotors, CMV promotors (e.g., immediate early CMV promotors), and AAV promoters. In bacterial systems, a strain of Escherichia coli such as BL-21 can be used.

Suitable E. coli vectors include the pGEX series of vectors (Amersham Biosciences Corp., Piscataway, NJ) that produce fusion proteins with glutathione S-transferase (GST). Transformed E. coli typically are grown exponentially, and then stimulated with isopropylthiogalactopyranoside (IPTG) prior to harvesting. In general, such fusion proteins can be soluble and can be purified from lysed cells by adsorption to glutathione-agarose beads followed by elution in the presence of free glutathione. The pGEX vectors can be designed to include thrombin or factor Xa protease cleavage sites so that the cloned target gene product can be released from the GST moiety.

A nucleic acid encoding a TRAILshort polypeptide or variant TRAILshort polypeptide provided herein can be cloned into, for example, a baculoviral vector such as pBlueBac (Invitrogen, Carlsbad, CA) and then used to co-transfect insect cells such as Spodoptera frugiperda (Sf9) cells with wild type DNA from Autographa californica multiply enveloped nuclear polyhedrosis virus (AcMNPV). Recombinant viruses producing polypeptides provided herein can be identified by standard methodology. In some cases, a nucleic acid encoding a TRAILshort polypeptide or variant TRAILshort polypeptide provided herein can be introduced into a SV40, retroviral, or vaccinia based viral vector and used to infect suitable host cells.

Any appropriate method can be used to make a nucleic acid molecule (or vector such as a plasmid vector or viral vector) having a nucleic acid sequence encoding a TRAILshort polypeptide or variant TRAILshort polypeptide provided herein. For example, molecule cloning techniques can be used to make a nucleic acid molecule (or vector such as a plasmid vector or viral vector) having a nucleic acid sequence encoding a TRAILshort polypeptide or variant TRAILshort polypeptide provided herein as described elsewhere (see, e.g., Sambrook et al., Molecular Cloning: A Laboratory Manual, 2nd edition, Cold Spring Harbor Laboratory, NY (1989); and Ausubel et al., Current Protocols in Molecular Biology, Green Publishing Associates and John Wiley & Sons, New York, N.Y. (1994)).

This document also provides host cells that include a nucleic acid provided herein (e.g., a nucleic acid having a nucleic acid sequence encoding a TRAILshort polypeptide or variant TRAILshort polypeptide provided herein). Host cells that can be designed to include one or more nucleic acids provided herein can be prokaryotic cells or eukaryotic cells. Examples of cells that can be designed to include a nucleic acid provided herein include, without limitation, E. coli (e.g., Tb-1, TG-1, DH5a, XL-Blue MRF (Stratagene), SA2821, or Y1090 cells), Bacillus subtilis, Salmonella typhimurium, Serratia marcescens, or Pseudomonas (e.g., P. aerugenosa) cells. Examples of eukaryotic cells that can be designed to include a nucleic acid provided herein include, without limitation, insect cells (e.g., Sf9 or Ea4 cells), yeast cells (e.g., S. cerevisiae cells), and mammalian cells (e.g., mouse, rat, hamster, monkey, or human cells). For example, VERO cells, HeLa cells, 3T3 cells, Chinese hamster ovary (CHO) cells, W138 BHK cells, COS-7 cells, and MDCK cells can be designed to include a nucleic acid provided herein. Any appropriate method can be used to introduce one or more nucleic acids provided herein (e.g., a vector such as a plasmid vector or viral vector having a nucleic acid sequence encoding a TRAILshort polypeptide or variant TRAILshort polypeptide) into a host cell. For example, calcium chloride-mediated transformation, transduction, conjugation, triparental mating, DEAE, dextran-mediated transfection, infection, membrane fusion with liposomes, high velocity bombardment with DNA-coated microprojectiles, direct microinjection into single cells, electroporation, or combinations thereof can be used to introduce a nucleic acid provided herein into a host cell (see, e.g., Sambrook et al., Molecular Biology: A Laboratory Manual, Cold Spring Harbor Laboratory, NY (1989); Davis et al., Basic Methods in Molecular Biology (1986); and Neumann et al., EMBO J., 1:841 (1982)).

As discussed herein, a TRAILshort polypeptide or variant TRAILshort polypeptide provided herein can be modified to contain an amino acid sequence that allows the polypeptide to be captured onto an affinity matrix. For example, a tag such as c-myc, hemagglutinin, polyhistidine, or Flag™ (Kodak) can be used to aid polypeptide purification. Such tags can be inserted anywhere within a polypeptide including at either the carboxyl or amino terminus. Other fusions that can be useful include enzymes that aid in the detection of a polypeptide, such as alkaline phosphatase. Immunoaffinity chromatography also can be used to purify a TRAILshort polypeptide or variant TRAILshort polypeptide provided herein.

In some cases, a TRAILshort polypeptide or variant TRAILshort polypeptide provided herein can be produced using a method that includes (a) introducing nucleic acid encoding the polypeptide into a host cell; (b) culturing the host cell in culture medium under conditions sufficient to express the polypeptide; (c) harvesting the polypeptide from the cell or culture medium; and (d) purifying the polypeptide (e.g., to reach at least 50, 60, 70, 80, 90, 95, 97, 98, or 99 percent purity).

A TRAILshort polypeptide or variant TRAILshort polypeptide provided herein can be purified by known chromatographic methods including DEAE ion exchange, gel filtration, and hydroxylapatite chromatography. See, e.g., Van Loon and Weinshilboum, Drug Metab. Dispos., 18:632-638 (1990); and Van Loon et al., Biochem. Pharmacol., 44:775-785 (1992).

A TRAILshort polypeptide, variant TRAILshort polypeptide, and/or TRAILshort peptidomimetic provided herein can be formulated as a composition for administration to a mammal (e.g., a human) having excessive T cell activation (e.g., a mammal having an autoimmune disorder, graft versus host disease, or lichen planus). For example, a TRAILshort polypeptide, variant TRAILshort polypeptide, and/or TRAILshort peptidomimetic provided herein can be formulated as a composition (e.g., a pharmaceutical composition) for administration to a mammal (e.g. a human) to reduce excessive T cell activation within the mammal and/or to reduce the symptoms associated with excessive T cell activation in the mammal. For example, a TRAILshort polypeptide, variant TRAILshort polypeptide, and/or TRAILshort peptidomimetic provided herein can be formulated with one or more pharmaceutically acceptable carriers (additives) and/or diluents. A pharmaceutical composition can be formulated for administration in solid or liquid form including, without limitation, sterile solutions, suspensions, sustained-release formulations, tablets, capsules, pills, powders, and granules.

In some cases, a composition for administration to a mammal (e.g., a human) having excessive T cell activation can include one or more additional bioactive molecules. In some cases, these molecules can be immunosuppressants. For example, a composition provided herein for administration to a mammal (e.g., a human) can include one or more immunosuppressants selected from cyclosporin, tacrolimus, sirolimus, everolimus, mycophenolate, steroids, hydroxychloroquine, cyclophosphamides, Jak inhibitors, Stat inhibitors, azathioprine, OKT3, basiliximab, daclizumab, abatacept (Orencia), adalimumab (Humira), anakinra (Kineret), certolizumab (Cimzia), etanercept (Enbrel), golimumab (Simponi), infliximab (Remicade), ixekizumab (Taltz), natalizumab (Tysabri), rituximab (Rituxan), secukinumab (Cosentyx), tocilizumab (Actemra), ustekinumab (Stelara), vedolizumab (Entyvio), basiliximab (Simulect), and daclizumab (Zinbryta). In some cases, these molecules can be anti-inflammatory agents. For example, a composition provided herein for administration to a mammal (e.g., a human) can include one or more anti-inflammatory molecules selected from inhibitors of Receptor Interacting Serine/Threonine Kinase 1 (Ripk1), tumor necrosis factor (TNF), interleukin 6 (IL6), IL6 receptor (IL6R), interleukin 1 beta (IL-1b), platelet-activating factor (PAF), CD40 ligand (CD40L), interleukin 4 receptor (IL4R), Burton tyrosine kinase (BTK), TNF Superfamily member 4 (OX40L), Complement, interleukin 23 (IL23), interleukin 13 (IL13), interleukin 2 (IL2), and Rho Associated Coiled-Coil Containing Protein Kinase 2 (ROCK2). Suitable Ripk1 inhibitors include, without limitation, primidone, RP-L201, fostamatinib, and necrostatin-1. Suitable TNF inhibitors include, without limitation, adalimumab, adalimumab-abdm, adalimumab-adaz, adalimumab-atto, certolizumab, etanercept, etanercept-szzs, golimumab, infliximab, infliximab-adba, and infliximab-dyyb. Suitable IL6 inhibitors include, without limitation, siltuximab. Suitable IL6R inhibitors include, without limitation, sarilumab, tocilizumab, and satralizumab. Suitable IL-1b inhibitors include, without limitation, anakinra, canakinumab, and rilonacept. Suitable PTAF inhibitors include, without limitation, (1R)-1,2,2-trimethylpropyl (R)-methylphosphinate and rilapladib. Suitable CD40L inhibitors include, without limitation, ruplizumab, BMS-986004, SL-172154, VIB4920, BG9588, and SAR441344. Suitable IL4R inhibitors include, without limitation, dupilumab, AER001, and AMG 317. Suitable BTK inhibitors include, without limitation, ibrutinib, dasatinib, acalabrutinib, fostamatinib, zanubrutinib, abivertinib, vecabrutinib, tirabrutinib, spebrutinib, branebrutinib, fenebrutinib, and evobrutinib. Suitable OX40L inhibitors include, without limitation SL-279252 and amlitelimab SAR445229 (also known as KY1005). Suitable complement inhibitors include, without limitation, eculizumab, Berinert, Cinryze, Sutimlimab, etanercept, palivizumab, bevacizumab, ravulizumab, and vilobelimab. Suitable IL23 inhibitors include, without limitation, ustekinumab, guselkumab, tildrakizumab, risankizumab, and briakinumab. Suitable IL13 inhibitors include, without limitation, cintredekin besudotox, lebrikizumab, tralokinumab, dupilumab, and AER001. Suitable IL2 inhibitors include, without limitation, cefazolin and pseudoephedrine. Suitable ROCK2 inhibitors include, without limitation, fasudil, Y-27632, ripasudil, netarsudil, fostamatinib, and belumosudil.

Pharmaceutically acceptable carriers, fillers, and vehicles that may be used in a pharmaceutical composition described herein include, without limitation, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

In some cases, a composition (e.g., a pharmaceutical composition) provided herein can include a plurality of particles (e.g., microparticles and/or nanoparticles), wherein each particle (e.g., microparticle and/or nanoparticles) of the plurality includes a TRAILshort polypeptide, a variant TRAILshort polypeptide, and/or a TRAILshort peptidomimetic. In some cases, at least a portion of the TRAILshort polypeptide, variant of a TRAILshort polypeptide, or the TRAILshort peptidomimetic is present on the surface of the particles (e.g., microparticles and/or nanoparticles). The particles can be liposomes (e.g., pegylated liposomes); extracellular vesicles including exosomes (e.g., extracellular vesicles typically <100 nm and originating from the endosomal membrane) or microvesicles (e.g., extracellular vesicles typically >100 nm and originating from the plasma membrane); or nanoparticles. See, e.g., Tracey, et al., Biochem Soc. Trans., 49(5):2253-2269 (2021); and Batrakova and Kim, J. Control Release, 219:396-405 (2015). In some cases, extracellular vesicles containing a TRAILshort polypeptide and/or a variant TRAILshort polypeptide can be produced from a host cell expressing the TRAILshort polypeptide and/or variant TRAILshort polypeptide by ultracentrifugation, size-based isolation, immunoaffinity capture, or precipitation. See, e.g., Meng, et al., Drug Deliv., 27(1):585-598 (2020).

A pharmaceutical composition containing a TRAILshort polypeptide, variant TRAILshort polypeptide, and/or TRAILshort peptidomimetic (or a plurality of particles such as microparticles containing a TRAILshort polypeptide, variant TRAILshort polypeptide, and/or TRAILshort peptidomimetic) can be designed for oral or parenteral (including subcutaneous, intramuscular, intravenous, and intradermal) administration. When being administered orally, a pharmaceutical composition can be in the form of a pill, tablet, or capsule. Compositions suitable for parenteral administration include aqueous and non-aqueous sterile injection solutions that can contain anti-oxidants, buffers, bacteriostats, and solutes that render the formulation isotonic with the blood of the intended recipient. The formulations can be presented in unit-dose or multi-dose containers, for example, sealed ampules and vials, and may be stored in a freeze dried (lyophilized) condition requiring only the addition of the sterile liquid carrier, for example, water for injections, immediately prior to use. Extemporaneous injection solutions and suspensions may be prepared from sterile powders, granules, and tablets.

In some cases, a pharmaceutically acceptable composition including a TRAILshort polypeptide, variant TRAILshort polypeptide, and/or TRAILshort peptidomimetic (or a plurality of particles such as microparticles containing a TRAILshort polypeptide, variant TRAILshort polypeptide, and/or TRAILshort peptidomimetic) can be administered locally or systemically. For example, a composition provided herein can be administered locally by intravenous injection or blood infusion. In some cases, a composition provided herein can be administered systemically, orally, or by injection to a mammal (e.g., a human).

Effective doses can vary depending on the severity of the excessive T cell activation, the route of administration, the age and general health condition of the subject, excipient usage, the possibility of co-usage with other therapeutic treatments, and the judgment of the treating physician.

An effective amount of a composition containing a TRAILshort polypeptide, variant TRAILshort polypeptide, and/or TRAILshort peptidomimetic (or a plurality of particles such as microparticles containing a TRAILshort polypeptide, variant TRAILshort polypeptide, and/or TRAILshort peptidomimetic) can be any amount that reduces one or more symptoms of the condition being treated, without producing significant toxicity to the mammal. With excessive T cell activation, for example, an effective amount can reduce excessive T cell activation, excessive T cell proliferation, excessive T cell effector functions, and/or cytokine secretion, and reduce inflammation or other symptoms of excessive T cell activation. For example, an effective amount of a composition containing a TRAILshort polypeptide, variant TRAILshort polypeptide, and/or TRAILshort peptidomimetic (or a plurality of particles such as microparticles containing a TRAILshort polypeptide, variant TRAILshort polypeptide, and/or TRAILshort peptidomimetic) can be from about 0.25 mg/kg to about 100 mg/kg (e.g., from about 0.3 mg/kg to about 11 mg/kg, from about 1 mg/kg to about 10 mg/kg, from about 2 mg/kg to about 10 mg/kg, from about 5 mg/kg to about 10 mg/kg, from about 6 mg/kg to about 10 mg/kg, from about 6 mg/kg to about 8 mg/kg, or from about 7 mg/kg to about 9 mg/kg). In some cases, from about 100 mg to about 1000 mg (e.g., from about 100 mg to about 250 mg, from about 125 mg to about 275 mg, from about 250 mg to about 1000 mg, from about 300 mg to about 1000 mg, from about 400 mg to about 1000 mg, from about 100 mg to about 900 mg, from about 100 mg to about 800 mg, from about 400 mg to about 800 mg, or from about 500 mg to about 700 mg) of a TRAILshort polypeptide, variant TRAILshort polypeptide, and/or TRAILshort peptidomimetic can be administered to an average sized human (e.g., about 75-85 kg human) per administration (e.g., per daily or weekly administration) for about two to about twelve weeks. In some cases, a composition containing a TRAILshort polypeptide, variant TRAILshort polypeptide, and/or TRAILshort peptidomimetic (or a plurality of particles such as microparticles containing a TRAILshort polypeptide, variant TRAILshort polypeptide, and/or TRAILshort peptidomimetic) can be administered daily within one of these dose ranges for a period of time (e.g., 14 or 21 days) followed by a seven-day rest period.

If a particular mammal fails to respond to a particular amount of a composition, then the amount of the composition containing the TRAILshort polypeptide, variant TRAILshort polypeptide, and/or TRAILshort peptidomimetic can be increased by, for example, two fold. After receiving this higher amount, the mammal can be monitored for both responsiveness to the treatment and toxicity symptoms, and adjustments made accordingly. The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and severity of the condition may require an increase or decrease in the actual effective amount administered.

The frequency of administration of a composition containing a TRAILshort polypeptide, variant TRAILshort polypeptide, and/or TRAILshort peptidomimetic (or a plurality of particles such as microparticles containing a TRAILshort polypeptide, variant TRAILshort polypeptide, and/or TRAILshort peptidomimetic) can be any amount that reduces the symptoms of the condition being treated without producing significant toxicity to the mammal. For example, the frequency of administration of a composition containing a TRAILshort polypeptide, variant TRAILshort polypeptide, and/or TRAILshort peptidomimetic (or a plurality of particles such as microparticles containing a TRAILshort polypeptide, variant TRAILshort polypeptide, and/or TRAILshort peptidomimetic) can be from about once a day to about once a month (e.g., from about once a week to about once every other week). The frequency of administration of a composition containing a TRAILshort polypeptide, variant TRAILshort polypeptide, and/or TRAILshort peptidomimetic (or a plurality of particles such as microparticles containing a TRAILshort polypeptide, variant TRAILshort polypeptide, and/or TRAILshort peptidomimetic) can remain constant or can be variable during the duration of treatment. A course of treatment with a composition containing a TRAILshort polypeptide, variant TRAILshort polypeptide, and/or TRAILshort peptidomimetic (or a plurality of particles such as microparticles containing a TRAILshort polypeptide, variant TRAILshort polypeptide, and/or TRAILshort peptidomimetic) can include rest periods. As with the effective amount, various factors can influence the actual frequency of administration used for a particular application. For example, the effective amount, duration of treatment, use of multiple treatment agents, route of administration, and severity of the condition may require an increase or decrease in administration frequency.

An effective duration for administering a composition containing a TRAILshort polypeptide, variant TRAILshort polypeptide, and/or TRAILshort peptidomimetic provided herein (or a plurality of particles such as microparticles containing a TRAILshort polypeptide, variant TRAILshort polypeptide, and/or TRAILshort peptidomimetic) can be any duration that reduces the symptoms of the condition being treated within without producing significant toxicity to the mammal. In some cases, the effective duration can vary from several days to several months. In general, the effective duration can range from about six weeks to about six months. Multiple factors can influence the actual effective duration used for a particular treatment. For example, an effective duration can vary with the frequency of administration, effective amount, use of multiple treatment agents, route of administration, and severity of the condition being treated.

The invention will be further described in the following examples, which do not limit the scope of the invention described in the claims.

### EXAMPLES

### Example 1—Methods for Biological Analysis

Western Blot: Peripheral CD3+ T cells were isolated from uninfected donor(s) and incubated overnight in RPMI containing decomplemented 1% FBS and 20 U/mL IL2. Culture plates were coated overnight using a solution of bicarbonate buffer containing 5 μg/mL TRAILshort peptide or BSA at 4° C. Cells were then incubated on the TRAILshort/BSA coated plates for 1 hour, followed by stimulation with CD3/CD28 Dynabeads (beads:cells=1:2) (Invitrogen) for 30 minutes. In parallel experiments, cells were treated for 3 hours with extracellular vesicles (EVs) containing TRAILshort (TsEV) or a variant of TRAILshort lacking the transmembrane domain encompassing amino acids 18-38 (TsΔTmEV), at a 1:1000 ratio of cell:EV, followed by CD3/CD28 beads for 30 minutes. Treated cells from both conditions were then collected, lysed with lysis buffer containing 50 mM Tris-HCl (pH 7.5), 150 mM NaCl, 1% NP-40, a protease inhibitor cocktail (Roche), and a phosphatase inhibitor (Thermo Fischer Scientific), and then used for protein assay. Cell lysates containing equal protein concentrations were run on SDS-PAGE gel and probed with anti-human antibodies against T cell receptor− (TCR−) associated signaling molecules, including ZAP70, phospho(p)-ZAP70 (Y319), LAT, p-LAT (Y191), p-LAT(Y220), Syk, p-Syk (T525/526), PLC7, p-PLC7 (Y783), p-SLP-76 (S376), LcK, p-LcK (Y505), and GAPDH. All antibodies were from Cell Signalling Technology.

Flow cytometry: Peripheral blood mononuclear cells (PBMCs) were obtained from uninfected donors and used to enrich CD4+ or CD8+ T cells by negative selection enrichment kits (STEMCELL™ Technologies). After overnight incubation in full RPMI containing 10% FBS and supplemented with 50 U/mL IL2, cells were simultaneously treated with EVs (TsEV or TsΔTmEV at 1:1000 cell:bead ratio) and stimulated with CD3/CD28 Dynabeads (beads:cells=1:2) for 24 hours, followed by flow cytometry analysis to detect the intracellular level of phosphorylated ZAP70 (Y319) (cell signaling). Briefly, treated cells were fixed with 4% paraformaldehyde (PFA) for 15 minutes at room temperature (RT), followed by permeabilization using 90% ice-cold methanol for 10 minutes on ice. Cells were then washed and incubated with primary anti human p-ZAP70 (Y319) antibody (1:1000) for 1 hour and then with secondary AF488-conjugated anti-rabbit IgG (H+L) Fab2 Fragment antibody (1:1000) for 30 minutes, both at RT. Flow cytometry data were acquired using BD FACSCANTO™ X and analyzed with the FlowJo(V10) software tool.

ELISpot Assay: PBMCs were isolated from peripheral blood of uninfected donors or HIV-infected donors using FICOLL-PAQUE® density gradient centrifugation. CD3+ or CD8+ T cells were enriched from PBMCs using negative selection enrichment kits (STEMCELL™ Technologies) and cultured overnight in full RPMI media containing 10% FBS and 50 U/mL IL-2 (Biolegend). 96-well culture plates were coated with 5 μg/mL of TRAILshort peptide or 0.1% BSA in PBS as a control and incubated overnight at 4° C. Isolated cells were cultured on the coated plates at a density of 5×10⁴ to 10×10⁴ cells/well/200 μL media, in the presence of 1:2 (bead:cell ratio) activation CD3/CD28 Dynabeads (Invitrogen) for 24 hours. Beads were then removed, and the cells were cultured on ELISpot plates (MAHAS4510) coated with anti-human IFNγ antibody (MABTECH). Uninfected cells were then treated with 5 μg/mL tetanus toxoid (HemaCare/Cellero), while HIV-infected cells were treated with 60 ng/mL of HIV-peptide mixture (Nef, Gag, Pol: NIH HIV Reagent Program), for 24 hours. ELISpot assay was performed using an established ELISA-based protocol. Briefly, plates were washed to remove the cells and incubated first with 2 μg/mL biotinylated anti-human IFNγ antibody (MABTECH) for 2 hours, washed 3× with wash buffer and then incubated with Avidin-Horseradish Peroxidase (HRP) (Invitrogen, 1:2000 dilution) for one hour. Plates were then incubated with AEC Substrate Solution (BD™ ELISPOT) and upon development of visible spots, the reaction was stopped using running water. After drying, plates were scanned, and the developed spots were counted using an ELISpot reader (Cellular Technology Limited; CTL). In a parallel and similar assay settings, instead of TRAILshort peptide/BSA, cells were treated with TsEV or TsΔTmEV, at a ratio of 1:1000 Cell/EV ratio. To assess the neutralizing effect of TRAILshort antibodies on IFNΥ secretion, a group of uninfected CD3+ cells were treated with 5 μg/mL of humanized anti Ts antibody (Ts Ab, clone: HC2LC3V3; Fusion Antibodies) for 24 hours, followed by ELISpot assay as described above.

Spleen cells were obtained from Marginal Zone Lymphoma (MZL) or Diffuse Large B-cell Lymphoma (DLBCL) patients and utilized for isolation of CD3+ T cells or CD19+ tumor B cells using appropriate enrichment kits (STEMCELL™ Technologies). Lymphoma cancer cells express TRAILshort, evading the TRAIL-induced anti-apoptotic effect (Fatma et al., Clin Cancer Res. 2020, 26(21):5759-5771). Isolated spleen CD3+ T cells were incubated with 5 μg/mL of Ts antibody (HC2LC3V3) or corresponding Isotype control (IgG1) in the presence of CD3/CD28 Dynabeads (1:2 bead:cell ratio) for 24 hours. Beads were then removed, and cells were transferred to pre-coated ELISpot plates with IFNγ antibody, followed by addition of CD19+ tumor cells (as a recall antigen) to CD3+ cells. After 24 hours, IFNγ secretion was assessed using ELISpot assay, according to the protocol described above.

Pull down assay: Human primary CD3+ T cells were enriched using a negative selection kit (STEMCELL™ Technologies) from uninfected donors and cultured overnight in full RPMI containing 10% FBS and supplemented with 50U IL-2. 30×10⁶ cells were treated with TsΔTmEV or TsEV (1:1000 cell:EV ratio), in the presence or absence of CD3/CD28 beads (1:1 bead:cell ratio) for 30 minutes. Cells were then collected, washed 1× with PBS and lysed with lysis buffer containing 50 mM Tris-HCl (pH 7.5), 150 mM NaCl, 1% NP-40, and supplemented with phosphatase and protease inhibitors. Cell lysates containing 0.5 mg total protein were first pre-cleared with 20 μL A/G agarose (Santa Cruz) at 4° C. for 1 hour and then incubated with 20 μl agarose-conjugated anti-human CD3 zeta antibody (Santa Cruz, sc-1239) at 4° C. overnight. The immunoprecipitates were washed 3× with lysis buffer and eluted with 30 μL 0.1M glycine-HCl (pH 2.6). SDS-PAGE was run to detect the immunoprecipitated proteins, including ZAP70, p-ZAP70 (Tyr319) (Cell Signaling) and CD3 zeta (Santa Cruz).

### Example 2—Production of TRAILshort Containing Extracellular Vesicles

HeLa TRAIL knockout cells were seeded into T175 flasks, and after two days of culture (˜80% confluence) the cells were transfected with pRuby-empty vector, pRuby-TRAILshort ΔTm vector, or pRuby TRAILshort vector (15 μg/flask) using 30 μL PEI with complete media. After 24 hours, the culture media were replaced with FBS-free Dulbecco's Modified Eagle Medium (DMEM) and culture was continued for 72 hours. The media were collected and centrifuged at 3500 rpm for 30 minutes to removes any cell debris. This conditioned media was subjected to tangential flow filtration using a 100 kDa filter. The samples were reduced to 30 mL after filtration. All samples were then lyophilized. Samples were analyzed via dynamic light scattering using the NanoSight NS300 instrument. Purity of the exosome also was assessed by western blot for CD9 and CD63 expression.

### Example 3—Ts and TsEV Reduce Phosphorylation Induced by TCR Signaling

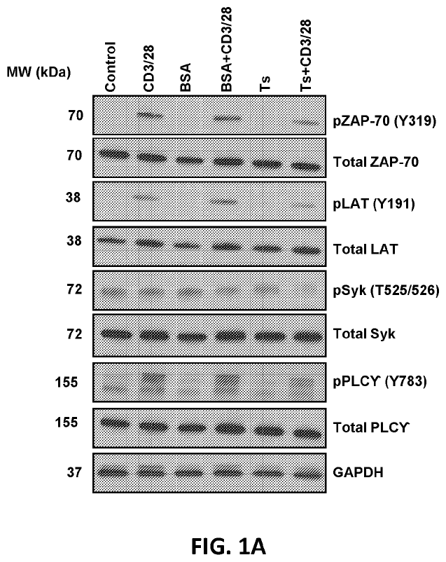

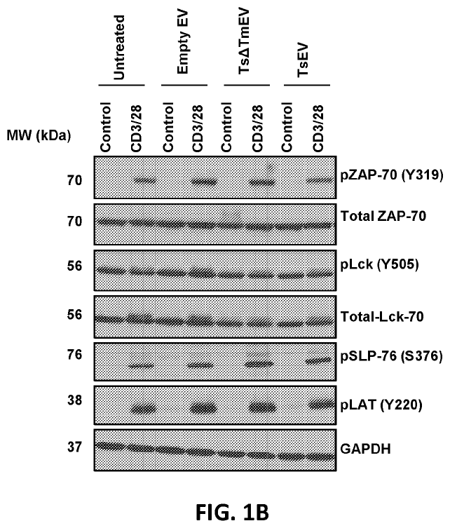

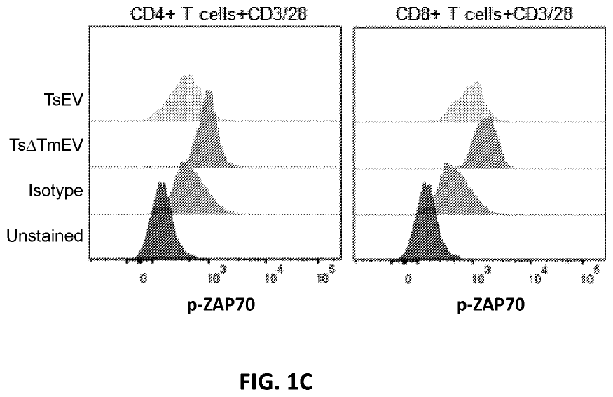

CD3+ T cells were isolated from uninfected donors and rested overnight in RPMI media containing 1% FBS and IL2. The rested cells were added to plates that had been coated with TRAILshort peptide (101 amino acids, SEQ ID NO:1) or BSA. After an hour, cells were stimulated with CD3/CD28 beads (1 bead:2 cells) for 30 minutes, followed by western blot analysis to detect TCR-associated signaling molecules (FIG. 1A). Pretreatment of cells with Ts peptide resulted in less phosphorylation of ZAP70 at position 319, less phosphorylation of Lat at position 191, less phosphorylation of Syk at position 535/536, and less phosphorylation of PLC gamma at position 783, as compared to levels of the phosphorylated proteins after CD3/CD28 treatment alone or after pretreatment with BSA.

In other studies, following incubation with RPMI media containing 1% FBS and IL2 overnight, cells were treated with TsEVs, TsΔTmEVs, or Empty EVs for three hours, followed by western blot analysis to detect TCR-associated signaling molecules (FIG. 1B). Pretreatment of cells with TsEV resulted in less phosphorylation of ZAP70 at position 319, less phosphorylation of LAT at position 220, and less phosphorylation of SLP76 at position 376, as compared to CD3/CD28 treatment alone or pretreatment with TsΔTmEV.

Enriched CD3⁺ cells (that also contain CD4⁺ and CD8⁺ T cells) were simultaneously stimulated with CD3/CD28 beads and treated with either TsEVs or TsΔTmEVs for 24 hours, followed by p-ZAP70 (Y319) detection by flow cytometry, as well as surface staining for CD4 and CD8 (FIG. 1C). Unstained cells and isotype control cells were performed on CD3 cells that contained a mixture of CD4 and CD8 cells. Phospho ZAP70 (at position 319) was reduced in TsEV-treated cells, but not in TsΔTmEV-treated cells.

### Example 4—Impact of TRAILshort on Antigen Recall

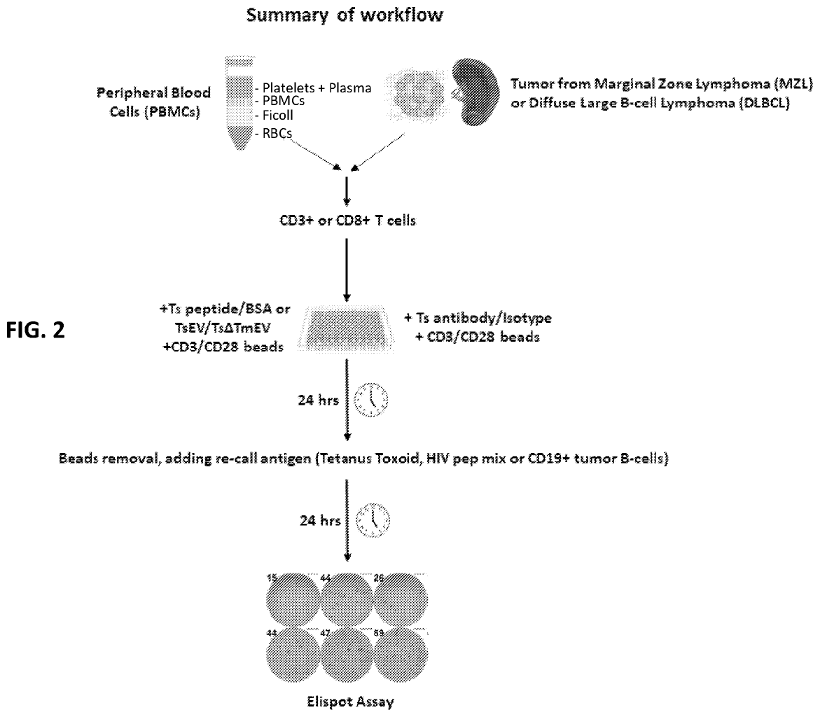

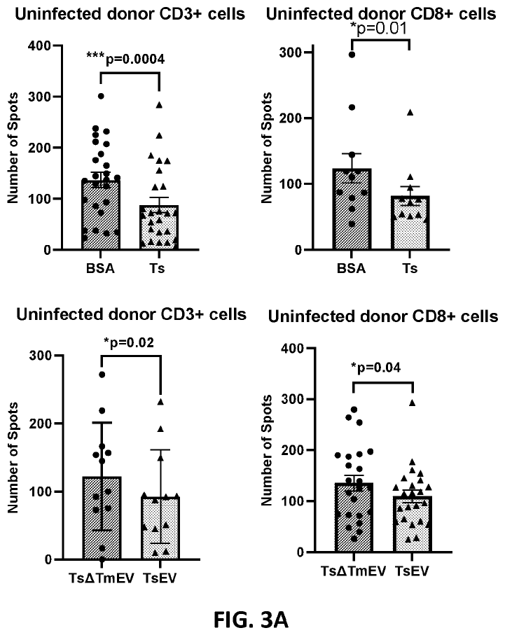

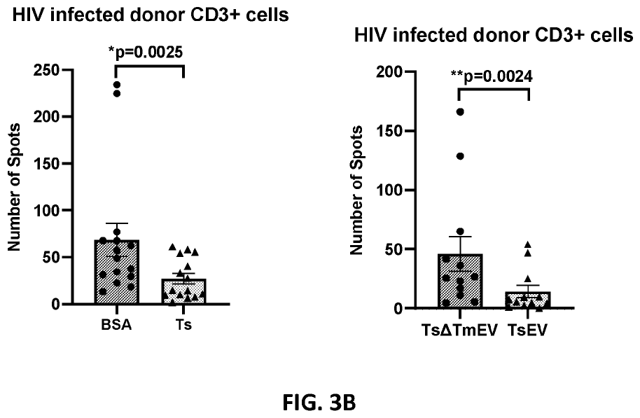

To evaluate the effect of Ts polypeptide, inhibitory anti-Ts antibody, TsEVs, and TsΔTmEV on the response of cells to either tetanus toxoid, an HIV peptide mixture (a combination of HIV-1 gag/pol and nef antigens), or autologous, malignant CD19+ tumor B-cells, an ELISpot assay was performed following the general protocol illustrated in FIG. 2 to detect antigen induced production of IFN7. Ts and TsEVs both inhibited CD3⁺ T cell and CD8⁺ T cell recall response to tetanus toxoid in cells from healthy donors. However, TsΔTmEV did not inhibit CD3⁺ T cell or CD8⁺ T cell recall response to the same stimulus (FIG. 3A). Similarly, Ts and TsEVs both inhibited recall response to an HIV peptide mixture by CD3⁺ T cells collected from HIV infected patients, but TsΔTmEV did not demonstrate any inhibition under identical conditions (FIG. 3B).

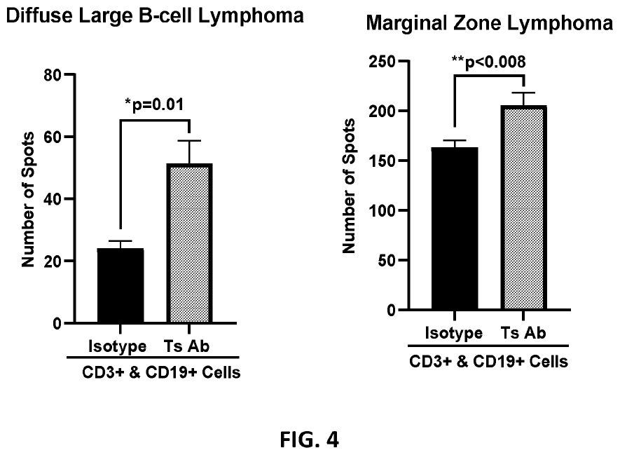

CD3⁺ T cells isolated from lymphoma patients (either diffuse large B-cell lymphoma or marginal zone lymphoma) were treated with isotype or Ts antibodies and evaluated for antigen recall in response to malignant CD19⁺ tumor B-cells. The Ts antibodies enhanced IFNγ production from CD3 T cells (FIG. 4).

Taken together, these results demonstrated the inhibitory activity of TRAILshort against T cell antigen response across a range of stimuli, and the ability of TRAILshort antibody to reverse that defect.

### Example 5—TsEV Reduces the Interaction of Phospho ZAP70 with the CD3 Zeta Chain Following CD3/CD28 Activation

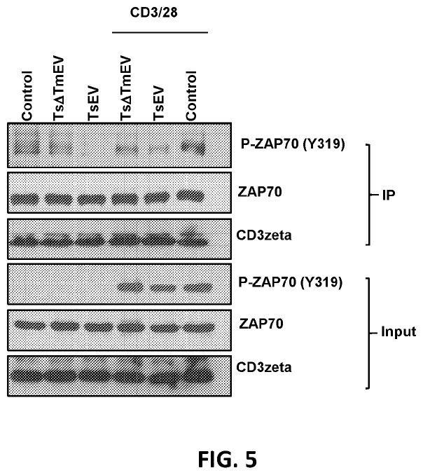

Primary CD3⁺ T cells from healthy patients were cultured overnight in full RPMI media containing IL-2, followed by treatment with either TsΔTmEV or TsEV in the presence or absence of CD3/CD28 beads for 30 minutes. Cells were collected, lysates were prepared, and the lysates were subjected to anti-CD3zeta immunoprecipitation. The immunoprecipitates were used in western blotting studies to detect co-immunoprecipitated proteins/phospho proteins that had a direct interaction with CD3zeta, including total and phosphorylated ZAP70 (Tyr319), using corresponding antibodies. In parallel, whole cell lysates were blotted for the same proteins. These studies demonstrated that TsEV reduced the amount of phosphorylated ZAP70 at position 319, which was associated with CD3 zeta following TCR activation by CD3/CD28 beads (FIG. 5).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for reducing excessive T cell activation in a mammal in need thereof, wherein said method comprises administering, to said mammal, a composition comprising a TRAILshort polypeptide, a variant TRAILshort polypeptide, or a TRAILshort fusion polypeptide, wherein T cell activation is reduced within said mammal.

2. The method of claim 1, wherein said mammal is a human.

3. The method of claim 1, wherein said mammal has an autoimmune disease, graft versus host disease, or lichen planus.

4. The method of claim 1, wherein said mammal was identified as having an autoimmune disease, graft versus host disease, or lichen planus prior to said administering step.

5. The method of claim 3, wherein said mammal has said autoimmune disease, and wherein said autoimmune disease is multiple sclerosis, rheumatoid arthritis, type 1 diabetes, systemic lupus erythematosus, Addison's disease, Graves' disease, Sjogren's syndrome, Hashimoto's thyroiditis, autoimmune vasculitis, organ transplant rejection, Celiac disease, pernicious anemia, psoriatic arthritis, or psoriasis.

6. A method for treating (a) an autoimmune disease in a mammal in need thereof, (b) graft versus host disease in a mammal in need thereof, or (c) lichen planus in a mammal in need thereof, wherein said method comprises administering, to said mammal, a composition comprising a TRAILshort polypeptide, a variant TRAILshort polypeptide, or a TRAILshort fusion polypeptide, wherein excessive T cell activation is reduced within said mammal, thereby treating said autoimmune disease, said graft versus host disease, or said lichen planus.

7-8. (canceled)

9. The method of claim 1, wherein said composition comprises said TRAILshort polypeptide, and wherein said TRAILshort polypeptide comprises the amino acid sequence of SEQ ID NO:1.

10. The method of claim 9, wherein said TRAILshort polypeptide is lacking a transmembrane domain.

11. The method of claim 9, wherein said TRAILshort polypeptide has the amino acid sequence of SEQ ID NO:2.

12. The method of claim 1, wherein said composition comprises said variant TRAILshort polypeptide, and wherein said variant TRAILshort polypeptide comprises a polypeptide having the amino acid sequence set forth in SEQ ID NO:1, where the amino acid residue at position 43 is replaced with an arginine residue, the amino acid residue at position 81 is replaced with a histidine residue, and/or the amino acid residue at position 85 is replaced with a lysine residue.

13. The method of claim 1, wherein said composition comprises said TRAILshort fusion polypeptide, wherein said TRAILshort fusion polypeptide comprises an affinity tag and a TRAILshort polypeptide or variant TRAILshort polypeptide.

14. The method of claim 13, wherein said affinity tag is a polyhistidine tag or a c-myc tag.

15. The method of claim 1, wherein said composition comprises a plurality of particles, wherein each particle of said plurality comprises said TRAILshort polypeptide, said variant TRAILshort polypeptide, or said TRAILshort fusion polypeptide.

16. The method of claim 15, wherein said particles are microparticles, exosomes, nanoparticles, or extracellular vesicles.

17. The method of claim 15, wherein at least a portion of said TRAILshort polypeptide, said variant of a TRAILshort polypeptide, or said TRAILshort fusion polypeptide is present on the surface of said particles.

18. A pharmaceutical composition comprising a plurality of particles, wherein each particle of said plurality comprises a TRAILshort polypeptide, a variant TRAILshort polypeptide, or a TRAILshort fusion polypeptide.

19. The composition of claim 18, wherein at least a portion of said TRAILshort polypeptide, said variant of a TRAILshort polypeptide, or said TRAILshort fusion polypeptide is present on the surface of said particles.

20-23. (canceled)

24. The composition of claim 18, wherein said composition further comprises an anti-inflammatory agent or an immunosuppressant.

25. The composition of claim 24, wherein said composition comprises said anti-inflammatory agent, and wherein said anti-inflammatory agent is selected from the group consisting of inhibitors of Receptor Interacting Serine/Threonine Kinase 1 (Ripk1), inhibitors of tumor necrosis factor (TNF), inhibitors of interleukin 6 (IL6), inhibitors of IL6 receptor (IL6R), inhibitors of interleukin 1 beta (IL-1b), inhibitors of platelet-activating factor (PAF), inhibitors of CD40 ligand (CD40L), inhibitors of interleukin 4 receptor (IL4R), inhibitors of Burton tyrosine kinase (BTK), inhibitors of TNF Superfamily member 4 (OX40L), inhibitors of Complement, inhibitors of interleukin 23 (IL23), inhibitors of interleukin 13 (IL13), inhibitors of interleukin 2 (IL2), and inhibitors of Rho Associated Coiled-Coil Containing Protein Kinase 2 (ROCK2).

26. (canceled)

27. The method of claim 24, wherein said composition comprises said immunosuppressant agent, and wherein said immunosuppressant is selected from the group consisting of cyclosporin, tacrolimus, sirolimus, everolimus, mycophenolate, steroids, hydroxychloroquine, cyclophosphamides, Jak inhibitors, Stat inhibitors, azathioprine, OKT3, basiliximab, daclizumab, abatacept, adalimumab, anakinra, certolizumab, etanercept, golimumab, infliximab, ixekizumab, natalizumab, rituximab, secukinumab, tocilizumab, ustekinumab, vedolizumab, basiliximab, and daclizumab.
